(19) United States
(12) Patent Application Publication
(10) Pub. No.: US 2011/0237535 A1
(43) Pub. Date: Sep. 29, 2011

(54) Use of Induced Pluripotent Cells and other Cells for Screening Compound Libraries

(75) Inventors: Alexander Yuzhakov, West Roxbury, MA (US); Eric Sandberg, DuPont, WA (US); Steven Shamah, Acton, MA (US); Rick Wagner, Cambridge, MA (US)

(73) Assignee: SRU Biosystems, Inc.

(21) Appl. No.: 13/073,233

(22) Filed: Mar. 28, 2011

Related U.S. Application Data
(60) Provisional application No. 61/317,995, filed on Mar. 26, 2010.
(60) Provisional application No. 61/323,076, filed on Apr. 12, 2010.
(60) Provisional application No. 61/363,824, filed on Jul. 13, 2010.

(51) Int. Cl. A61K 31/704; C12Q 1/02; C12Q 1/68; A61K 31/138; A61K 31/4412; A61K 31/135

(52) U.S. Cl. 514/34; 435/29; 435/6.1; 514/651; 514/348; 514/656

(57) ABSTRACT

The invention provides methods for screening test compounds or toxins for effects on cells. The invention also provides methods for determining frequency, amplitude and kinetic profiles of cells.

### PRIORITY

This application claims the benefit of U.S. Ser. No. 61/317,995, which was filed on Mar. 26, 2010, U.S. Ser. No. 61/323,076, which was filed on Apr. 12, 2010, and U.S. Ser. No. 61/363,824, which was filed on Jul. 13, 2010. All of these applications are incorporated by reference herein in their entirety.

### SUMMARY OF THE INVENTION

One embodiment of the invention provides a method for screening a compound or environmental condition for an effect on cells, cell aggregates, or tissue. The method comprises applying the cells, cell aggregates, or tissue to a colorimetric resonant reflectance biosensor surface, a dielectric film stack biosensor surface, or a grating-based waveguide biosensor surface. The cells, cell aggregates, or tissue are contacted with the compound or environmental condition and periodic or continuous peak wavelength values or effective refractive index values are detected during a time course. Peak wavelength values or effective refractive index values are analyzed for frequency, amplitude, or kinetic profile, or a combination thereof over the time course. A change in frequency, amplitude, or kinetic profile after the compound or environmental condition is contacted with the cells, cell aggregates, or tissue indicates that the compound or environmental condition has an effect on the cells, cell aggregates, or tissue. Optionally, two or more concentrations of the compound can be added to one or more populations the cells, cell aggregates, or tissue at two or more distinct locations on the biosensor surface. The cells can be stem cells, human or mammalian induced pluripotent stem cells, cells differentiated from the human or mammalian induced pluripotent cells, neural stem cells, neurons, cardiomyocyte stem cells, cardiomyocytes, hepatic stem cells, hepatocytes or combinations thereof. The human or mammalian induced pluripotent stem cell line or cells differentiated from the human or mammalian induced pluripotent cells can be cardiomyocytes. The peak wavelength values or effective refractive index values can be analyzed for frequency or amplitude, wherein a decreased frequency over the time course of the assay indicates a negative effect of the compound or environmental condition on the cells, cell aggregates, or tissue, and wherein a decreased amplitude over the time course of the assay indicates a negative effect of the compound or environmental condition on the cells, cell aggregates or tissue. The peak wavelength values or effective refractive index values can be analyzed for frequency or amplitude, wherein a decreased frequency with increasing compound concentration indicates a negative effect of the compound on the cells, cell aggregates, or tissue and wherein a decreased amplitude with increasing compound concentration indicates a negative effect of the compound or environmental condition on the cells, cell aggregates, or tissue. The peak wavelength values can be analyzed for kinetic profile, wherein a kinetic profile that moves from a positive peak wavelength value to a negative peak wavelength value over the time course indicates a negative effect of the compound or environmental condition on the cells, cell aggregates, or tissue. The peak wavelength values can be analyzed for kinetic profile, wherein a kinetic profile that moves from a positive peak wavelength value to a negative peak wavelength value with increasing concentration of the compound indicates a negative effect of the compound or environmental condition on the cells, cell aggregates, or tissue. The compound can be a drug, a calcium channel blocker, a β-adrenoreceptor agonist, an α-adrenoreceptor agonist, a test reagent, a polypeptide, a polynucleotide, a modifier of a hERG channel, or a toxin. The cell aggregates can be embroid bodies. The cells, cell aggregates, or tissue can be further contacted with at least one second compound or second environmental condition in the presence of the first compound or first environmental condition.

Another embodiment of the invention provides a method for reducing the risk of pharmacological agent toxicity in a subject. The method comprises contacting one or more cells differentiated from an induced pluripotent stem cell line generated from the subject with a dose of a pharmacological agent. The contacted one or more cells are assayed for toxicity by applying the cells to a colorimetric resonant reflectance biosensor surface, a dielectric film stack biosensor surface, or a grating-based waveguide biosensor surface and contacting the cells with the pharmacological agent. Periodic or continuous peak wavelength values or effective refractive index values are detected during a time course. The peak wavelength values or effective refractive index values are analyzed for frequency, amplitude, or kinetic profile or a combination thereof over the time course. A negative change in frequency, amplitude, or kinetic profile after the pharmacological agent is contacted with the cells indicates that the pharmacological agent has a negative toxicity effect on the cells. The pharmacological agent is prescribed or administered to the subject only if the pharmacological agent does not have a negative toxicity effect on the contacted cells, thereby reducing the risk of pharmacological toxicity in a subject.

Even another embodiment of the invention provides a method for reducing the risk of pharmacological agent toxicity in a subject. The method comprises contacting one or more cell populations differentiated from an induced pluripotent stem cell line generated from the subject with two or more dose concentrations of a pharmacological agent. The contacted one or more cell populations are assayed for toxicity by applying the one or more cell populations to a colorimetric resonant reflectance biosensor surface, a dielectric film stack biosensor surface, or a grating-based waveguide biosensor surface and contacting the one or more cell populations with two of more concentrations the pharmacological agent. One or more peak wavelength values or effective refractive index values are detected for each concentration of the pharmacological agent. The peak wavelength values or effective refractive index values are analyzed for frequency, amplitude, or kinetic profile or a combination thereof for each concentration of the pharmacological agent. A negative change in frequency, amplitude, or kinetic profile after the pharmacological agent is contacted with the cells indicates that the pharmacological agent concentration has a negative toxicity effect on the cells. The pharmacological agent is prescribed or administered to the subject only if the pharmacological agent concentration does not have a negative toxicity effect in the contacted cells, thereby reducing the risk of pharmacological toxicity in a subject.

Still another embodiment of the invention provides a method of screening a compound for neutralizing activity on a toxin or negative environmental condition. The method comprises applying cells, cell aggregates, or tissue to a colorimetric resonant reflectance biosensor surface, a dielectric film stack biosensor surface, or a grating-based waveguide biosensor surface and contacting the cells, cell aggregates, or tissue with the toxin or negative environmental condition and the compound. Periodic or continuous peak wavelength values or effective refractive index values are detected during a time course. The peak wavelength values or effective refractive index values are analyzed for frequency, amplitude, or kinetic profile or a combination thereof over the time course. A positive change in frequency, amplitude, or kinetic profile after the compound is contacted with the cells, cell aggregates, or tissue indicates that the compound has a neutralizing effect on the toxin or negative environmental condition.

Yet another embodiment of the invention provides a method of screening a compound for neutralizing activity on a toxin or negative environmental condition. One or more cells, cell aggregates, or tissue populations are applied to a colorimetric resonant reflectance biosensor surface, a dielectric film stack biosensor surface, or a grating-based waveguide biosensor surface and contacted with the toxin or negative environmental condition and the compound at two or more compound concentrations. Periodic or continuous peak wavelength values or effective refractive index values are detected during a time course for each compound concentration. Peak wavelength values or effective refractive index values are analyzed for frequency, amplitude, or kinetic profile or a combination thereof for each compound concentration over the time course. A positive change in frequency, amplitude, or kinetic profile after the compound is contacted with the cells, cell aggregates, or tissue indicates that the compound has a neutralizing effect on the toxin.

Another embodiment of the invention provides a method of screening a test toxin for a signature kinetic profile to determine a class or subclass of the test toxin. The method comprises applying cells, cell aggregates, or tissue to a colorimetric resonant reflectance biosensor surface, a dielectric film stack biosensor surface, or a grating-based waveguide biosensor surface and contacting the cells, cell aggregates, or tissue with the test toxin. Periodic or continuous peak wavelength values or effective refractive index values are detected during a time course of the assay. The peak wavelength values or effective refractive index values are analyzed for frequency, amplitude, or kinetic profile or a combination thereof over the time course of the assay to generate a signature kinetic profile of the test toxin's effects on the cells, cell aggregates, or tissue. The signature kinetic profile of the test toxin is compared to signature kinetic profiles of known toxins, wherein the test toxin is placed into a class or subclass of toxins having a similar signature kinetic profile as the test toxin.

Even another embodiment of the invention provides a method for determining the effect of a test compound or environmental condition on the sinus rhythm of cardiomyocytes. The method comprises applying the cardiomyocytes to a colorimetric resonant reflectance biosensor surface, a dielectric film stack biosensor surface, or a grating-based waveguide biosensor surface and contacting the cardiomyocytes with the compound or environmental condition. Periodic or continuous peak wavelength values or effective refractive index values are detected during a time course. The peak wavelength values or effective refractive index values are analyzed for sinus rhythm over the time course. A change in the sinus rhythm after the compound or environmental condition is contacted with the cardiomyocytes indicates that the compound or environmental condition has an effect on the sinus rhythm of the cardiomyocytes. The effect of the test compound or environmental condition on the sinus rhythm can be a prolongation or shortening of the QT interval.

Another embodiment of the invention provides a method for determining a beat or burst pattern of cardiac or neuronal cells, cardiac or neuron cell aggregates, or cardiac or neuronal tissue. The method comprises applying the cells, cell aggregates, or tissue to a colorimetric resonant reflectance biosensor surface, a dielectric film stack biosensor surface, or a grating-based waveguide biosensor surface and detecting periodic or continuous peak wavelength values or effective refractive index values during a time course. The peak wavelength values or effective refractive index values are analyzed for frequency, amplitude, or kinetic profile, or a combination thereof over the time course. A beat or burst pattern of the cardiac or neuronal cells, cardiac or neuron cell aggregates, or cardiac or neuronal tissue is determined. Optionally, one or more compounds are added to the cells, cell aggregates, or tissue before or after they are applied to the biosensor surface.

### BRIEF DESCRIPTION OF THE DRAWINGS

FIG. 1 demonstrates the kinetic profile of two toxins on Vero cells.

FIG. 2 shows the differential neutralization of cytotoxicity in Vero cells by certain compounds.

FIG. 3A-B shows detection of toxin-induced cell death and antidote protection.

FIG. 3A shows the temporal response profile and FIG. 3B shows the results at an 11 hour time point.

FIG. 4 demonstrates that cytotoxic compounds with distinct mechanisms of action each have distinct kinetic profiles.

FIG. 5A-D show concentration dependent cytotoxicity for four toxins in HeLa cells.

FIG. 5A shows cycloheximide; FIG. 5B shows digitonin; FIG. 5C shows doxorubicin;

FIG. 5D shows tamoxifen.

FIG. 6 shows the kinetic profiles of tamoxifen, 4-hydroxy-tamoxifen, and raloxifene (unlabeled line).

FIG. 7 shows the kinetic profiles of DNA damaging agents over time.

FIG. 8A-B shows the kinetic profiles for differing concentrations of potassium dichromate (FIG. 8A) and cisplatin (FIG. 8B).

FIG. 9A-B shows the kinetic profile of vinblastine over early time points (FIG. 9B) and over long term time points (FIG. 9A).

FIG. 10A-B demonstrates dose-dependent toxicity of doxorubicin on murine embryonic stem cell-derived cardiomyocytes. FIG. 10A demonstrates the effect of doxorubicin over time. FIG. 10B demonstrates the effect of doxorubicin concentration.

FIG. 11 shows the detection of beating murine embryonic stem cell-derived cardiomyocytes and murine embryonic stem cell-derived cardiomyocytes treated with KCl.

FIG. 12 shows the detection of beating murine embryonic stem cell-derived cardiomyocytes.

FIG. 13 shows the detection of beating murine embryonic stem cell-derived cardiomyocytes and the inhibition of beating by KCl.

FIG. 14 shows the effect of doxorubicin or no treatment on beating cardiomyocytes at doxorubicin concentrations of 10 μM, 1 μM, 0.1 μM, 0.01 μM, and 0 μM.

FIG. 15A-D shows cardiomyocyte beating phenotypes. FIG. 15A shows high frequency beating, FIG. 15B shows moderate frequency beating, FIG. 15C shows slow frequency beating, and FIG. 15D shows irregular frequency beating.

FIG. 16A-D shows cardiomyocyte dense synchronous beating (FIG. 16A-B) and cardiomyocyte sparse asynchronous beating (FIG. 16C-D).

FIG. 17A-D shows the effect of amitriptyline on cardiomyocytes. FIG. 17A shows the cardiomyocytes prior to the addition of the amitriptyline. FIGS. 17B, 17C, and 17D show the cardiomyocytes over time at 6 minutes, 10 minutes, and 15 minutes, respectively, after the addition of amitriptyline.

### DETAILED DESCRIPTION OF THE INVENTION

As used herein, the singular forms “a,” “an,” and “the” include plural referents unless the context clearly dictates otherwise.

### Biosensors

Biosensors of the invention can be colorimetric resonant reflectance biosensors or grating-based waveguide biosensors. See e.g., Cunningham et al., “Colorimetric resonant reflection as a direct biochemical assay technique,” Sensors and Actuators B, Volume 81, p. 316-328, Jan. 5, 2002; U.S. Pat. Publ. No. 2004/0091397; U.S. Pat. No. 7,094,595; U.S. Pat. No. 7,264,973. Colorimetric resonant biosensors are not surface plasmon resonant (SPR) biosensors. SPR biosensors have a thin metal layer, such as silver, gold, copper, aluminum, sodium, and indium. The metal must have conduction band electrons capable of resonating with light at a suitable wavelength. A SPR biosensor surface exposed to light must be pure metal. Oxides, sulfides and other films interfere with SPR. Colorimetric resonant biosensors do not have a metal layer, rather they have a dielectric coating of high refractive index material, such as zinc sulfide, titanium dioxide, tantalum oxide, and silicon nitride.

Biosensors of the invention can also be dielectric film stack biosensors (see e.g., U.S. Pat. No. 6,320,991), diffraction grating biosensors (see e.g., U.S. Pat. Nos. 5,955,378; 6,100,991) and diffraction anomaly biosensors (see e.g., U.S. Pat. No. 5,925,878; RE37,473). Dielectric film stack biosensors comprise a stack of dielectric layers formed on a substrate having a grooved surface or grating surface (see e.g., U.S. Pat. No. 6,320,991). The biosensor receives light and, for at least one angle of incidence, a portion of the light propagates within the dielectric layers. The parameters of a sample medium are determined by detecting shifts in optical anomalies, i.e., shifts in a resonance peak or notch. Shifts in optical anomalies can be detected as either a shift in a resonance angle or a shift in resonance wavelength.

Other biosensors that can be used with the methods of the invention include grating-based waveguide biosensors, which are described in, e.g., U.S. Pat. No. 5,738,825. A grating-based waveguide biosensor comprises a waveguiding film and a diffraction grating that incouples an incident light field into the waveguiding film to generate a diffracted light field. A change in the effective refractive index of the waveguiding film is detected. Devices where the wave must be transported a significant distance within the device, such as grating-based waveguide biosensors, lack the spatial resolution of colorimetric resonant reflection biosensors.

A colorimetric resonant reflectance biosensor allows biochemical interactions to be measured on the biosensor's surface without the use of fluorescent tags, colorimetric labels or any other type of detection tag or detection label. A biosensor surface contains an optical structure that, when illuminated with collimated and/or white light, is designed to reflect or transmit only a narrow band of wavelengths (“a resonant grating effect”). For reflection the narrow wavelength band is described as a wavelength “peak.” For transmission the narrow wavelength band is described as a wavelength “dip.” The “peak wavelength value” (PWV) changes when materials, such as biological materials, are deposited or removed from the biosensor surface. Wavelength dips can also be detected. A readout instrument is used to illuminate distinct locations on a biosensor surface with collimated and/or white light, and to collect reflected light. The collected light is gathered into a wavelength spectrometer for determination of a PWV.

Wherever the changes in PWV is discussed herein, it is understood that shifts in resonance angle, shifts in resonance wavelength, and changes in effective refractive index can be substituted depending upon the type of biosensor used. Additionally, where colorimetric resonant reflectance biosensors are discussed herein, it is understood that dielectric film stack biosensors, diffraction grating biosensors, diffraction anomaly biosensors, and grating-based waveguide biosensors can be substituted.

A detection system can comprise a biosensor, a light source that directs light to the biosensor, and a detector that detects light reflected from the biosensor. In one embodiment, it is possible to simplify the readout instrumentation by the application of a filter so that only positive results over a determined threshold trigger a detection.

A light source can illuminate a colorimetric resonant reflectance biosensor from its top surface, i.e., the surface to which cells are applied or from its bottom surface. By measuring the shift in resonant wavelength at each distinct location of a biosensor, it is possible to determine which distinct locations have mass bound to or associated with them. The extent of the shift can be used to determine, e.g., the amount of ligands in a test sample or the chemical affinity between one or more specific binding substances and the ligands of the test sample. The extent of shift can also be used to detect small changes in mass on the sensor surface.

A biosensor can be illuminated twice. The first measurement determines the reflectance spectra of one or more distinct locations of a biosensor array with cells immobilized on the biosensor. The second measurement determines the reflectance spectra after one or more compounds are applied to a biosensor or a change in environmental conditions is made. The difference in peak wavelength between these two measurements is a measurement of the effect of the compounds or environmental conditions on the cells. This method of illumination can control for small nonuniformities in a surface of a biosensor that can result in regions with slight variations in the peak resonant wavelength. This method can also control for varying concentrations or molecular weights of cells on a biosensor.

A biosensor can be illuminated two or more times at two or more time points to create periodic peak wavelength readings (or other readings, e.g., shift in resonant angle readings, shift in wavelength readings, or refractive index readings). Alternatively, a biosensor can be continuously illuminated and readings collected continuously. A time course of an assay can be about 1/100, 1/10 or ½ of second, 1, 2, 5, 10, 20, 30, 45 or 60 seconds, 1, 2, 3, 4, 5, 10, 20, or 60 minutes, 2, 3, 4, 5, 12, 24, 36, 48, 72 hours or more.

Cells such as primary cells or stem cells can be immobilized to the biosensor by one or more ligands. In one embodiment of the invention, cells are immobilized to the biosensor through a reaction with extracellular matrix ligands. Integrins are cell surface receptors that interact with the extracellular matrix (ECM) and mediate intracellular signals. Integrins are responsible for cytoskeletal organization, cellular motility, regulation of the cell cycle, regulation of cellular of integrin affinity, attachment of cells to viruses, attachment of cells to other cells or ECM. Integrins are also responsible for signal transduction, a process whereby the cell transforms one kind of signal or stimulus into another intracellularly and intercellularly. Integrins can transduce information from the ECM to the cell and information from the cell to other cells (e.g., via integrins on the other cells) or to the ECM. A list of integrins and their ECM ligands can be found in Takada et al. Genome Biology 8:215 (2007) as shown in Table 1.

TABLE 1

| Integrin | ECM ligand |
|---|---|
| α₁β₁ | Laminin, collagen |
| α₂β₁ | Laminin, collagen, thrombospondin, E-cadherin, |
| | tenascin |
| α₃β₁ | Laminin, thrombospondin, uPAR |
| α₄β₁ | Thrombospondin, MadCAM-1, VCAM-1, |
| | fibronectin, osteopontin, ADAM, ICAM-4 |
| α₅β₁ | Fibronectin, osteopontin, fibrillin, |
| | thrombospondin, ADAM, COMP, L1 |
| α₆β₁ | Laminin, thrombospondin, ADAM, Cyr61 |
| α₇β₁ | Laminin |
| α₈β₁ | Tenascin, fibronectin, osteopontin, vitronectin, |
| | LAP-TGF-β, nephronectin, |
| α₉β₁ | Tenascin, VCAM-1, osteopontin, uPAR, plasmin, |
| | angiostatin, ADAM, VEGF-C, VEGF-D |
| α₁₀β₁ | Laminin, collegen |
| α₁₁β₁ | Collagen |
| αvβ₁ | LAP-TGF-β, fibronectin, osteopontin, L1 |
| αLβ₂ | ICAM, ICAM-4 |
| αMβ₂ | ICAM, iC3b, factor X, fibrinogen, ICAM-4, |
| | heparin |
| αXβ₂ | ICAM, iC3b, fibrinogen, ICAM-4, heparin, |
| | collagen |
| αDβ₂ | ICAM, VCAM-1, fibrinogen, fibronectin, |
| | vitronectin, Cyr61, plasminogen |
| α_{IIb}β₃ | Fibrinogen, thrombospondin, fibronectin, |
| | vitronectin, vWF, Cyr61, ICAM-4, L1, CD40 |
| | ligand |
| αᵥβ₃ | Fibrinogen, vitronectin, vWF, thrombospondin, |
| | fibrillin, tenascin, PECAM-1, fibronectin, |
| | osteopontin, BSP, MFG-E8, ADAM-15, COMP, |
| | Cyr61, ICAM-4, MMP, FGF-2, uPA, uPAR. L1, |
| | angiostatin, plasmin, cardiotoxin, LAP-TGF- β, |
| | Del-1 |
| α6β₄ | Laminin |
| αᵥβ₅ | Osteopontin, BSP, vitronectin, CCN3 [35], LAP- |
| | TGF- β |
| αᵥβ₆ | LAP-TGF- β, fibronectin, osteopontin, ADAM |
| α₄β₇ | MAdCAM-1, VCAM-1, fibronectin, osteopontin |
| αEβ₇ | E-cadherin |
| αvβ₈ | LAP-TGF- β |

Abbreviations:
ADAM, a disintegrin metalloprotease;
BSP, bone sialic protein;
CCN3, an extracellular matrix protein;
COMP, cartilage oligomeric matrix protein,
Cyr61, cysteine-rich protein 61;
L1, CD171;
LAP-TGF- β latency-associated peptide;
iC3b, inactivated complement component 3;
PECAM-1, platelet and endothelial cell adhesion molecule 1;
uPA, urokinase;
uPAR, urokinase receptor;
VEGF, vascular endothelial growth factor;
vWF, von Willebrand Factor.

Other cell surface receptors that interact with the ECM include focal adhesion proteins. Focal adhesion proteins form large complexes that connect the cytoskeleton of a cell to the ECM. Focal adhesion proteins include, for example, talin, α-actinin, filamin, vinculin, focal adhesion kinase, paxilin, parvin, actopaxin, nexilin, fimbrin, G-actin, vimentin, syntenin, and many others.

Yet other cell surface receptors can include, but are not limited to those that can interact with the ECM include cluster of differentiation (CD) molecules. CD molecules act in a variety of ways and often act as receptors or ligands for the cell. Other cell surface receptors that interact with ECM include cadherins, adherins, and selectins.

The ECM has many functions including providing support and anchorage for cells, segregation of tissue from one another, and regulation of intracellular communications. The ECM is composed of fibrous proteins and glycosaminoglycans. Glycosaminoglycans are carbohydrate polymers that are usually attached to the ECM proteins to form proteoglycans (including, e.g., heparin sulfate proteoglycans, chondroitin sulfate proteoglycans, karatin sulfate proteoglycans). A glycosaminoglycan that is not found as a proteoglycan is hyaluronic acid. ECM proteins include, for example, collagen (including fibrillar, facit, short chain, basement membrane and other forms of collagen), fibronectin, elastin, and laminin (see Table 1 for additional examples of ECM proteins). ECM ligands useful herein include ECM proteins and/or peptide fragments thereof (e.g. RGD-containing peptide fragments of fibronectin or peptide fragments of collagen), glycosaminoglycans, proteoglycans, and hyaluronic acid.

“Specifically binds,” “specifically bind” or “specific for” means that a cell surface receptor, e.g., an integrin or focal adhesion protein, etc., binds to a cognate extracellular matrix ligand, with greater affinity than to other, non-specific molecules. A non-specific molecule does not substantially bind to the cell receptor. For example, the integrin α4/β1 specifically binds the ECM ligand fibronectin, but does not specifically bind the non-specific ECM ligands collagen or laminin. In one embodiment of the invention, specific binding of a cell surface receptor to an extracellular matrix ligand, wherein the extracellular matrix ligand is immobilized to a surface, e.g., a biosensor surface, will immobilize the cell to the extracellular matrix ligand and therefore to the surface such that the cell is not washed from the surface by normal cell assay washing procedures.

By specifically immobilizing cells to a biosensor surface through binding of cell surface receptors, such as integrins, to ECM ligands that are immobilized to the biosensor, the binding of the cells to the biosensor and the cells' response to stimuli can be dramatically altered as compared to cells that are non-specifically immobilized to a biosensor surface. Although not required, detection of response of cells to stimuli can be greatly enhanced or augmented where cells are immobilized to a biosensor via ECM ligand binding. In one embodiment of the invention, the cells are in a serum-free medium. A serum-free medium contains about 10, 5, 4, 3, 2, 1, 0.5% or less serum. A serum-free medium can comprise about 0% serum or about 0% to about 1% serum. In one embodiment of the invention cells are added to a biosensor surface where one or more types of ECM ligands have been immobilized to the biosensor surface. In another embodiment of the invention, cells are combined with one or more types of ECM ligands and then added to the surface of a biosensor.

In one embodiment of the invention, an ECM ligand is purified. A purified ECM ligand is an ECM ligand preparation that is substantially free of cellular material, other types of ECM ligands, chemical precursors, chemicals used in preparation of the ECM ligand, or combinations thereof. An ECM ligand preparation that is substantially free of other types of ECM ligands, cellular material, culture medium, chemical precursors, chemicals used in preparation of the ECM ligand, etc., has less than about 30%, 20%, 10%, 5%, 1% or more of other ECM ligands, culture medium, chemical precursors, and/or other chemicals used in preparation. Therefore, a purified ECM ligand is about 70%, 80%, 90%, 95%, 99% or more pure. A purified ECM ligand does not include unpurified or semi-purified preparations or mixtures of ECM ligands that are less than 70% pure, e.g., fetal bovine serum. In one embodiment of the invention, ECM ligands are not purified and comprise a mixture of ECM proteins and non-ECM proteins. Examples of non-purified ECM ligand preparations include fetal bovine serum, bovine serum albumin, and ovalbumin.

A biosensor of the invention can comprise an inner surface, for example, a bottom surface of a liquid-containing vessel. A liquid-containing vessel can be, for example, a microtiter plate well, a test tube, a petri dish, or a microfluidic channel. One embodiment of this invention is a biosensor that is incorporated into any type of microtiter plate. For example, a biosensor can be incorporated into the bottom surface of a microtiter plate by assembling the walls of the reaction vessels over the biosensor surface, so that each reaction “spot” can be exposed to a distinct test sample. Therefore, each individual microtiter plate well can act as a separate reaction vessel. Separate chemical reactions can, therefore, occur within adjacent wells without intermixing reaction fluids and chemically distinct test solutions can be applied to individual wells.

### Cell Assays

Assays of the invention can provide more information than just a readout on cell death versus cells remaining alive. Assays of the invention can provide frequency, rate and kinetic profile information for cells in culture. In particular, assays of the invention can be used to measure the frequency, rate, and kinetic profile of beating cardiomyocytes or bursting neurons in culture. The ability of assays of the invention to measure the frequency and rate of beating cardiomyocytes or bursting neurons represents a new method for measuring cytotoxic or other effects. The ‘beating’ phenomenon in cardiomyocytes is typically measured one cell at a time using patch clamp methodology that measures the opening and closing of the hERG channel—a potassium ion channel that mediates the beating phenomenon. When the hERG channel is compromised, such as by an inhibitor of the channel, a long QT syndrome can develop, often leading to death. The assays of the invention allow for beating to be measured, not just simply the voltage potential changes across one or a few channels in a patch clamp system. Thus, the assays of the invention allow for detection of changes in beating frequency and beating rate via the hERG channel. Additionally, at the same time alternative ion channels to be measured, as an orthogonal approach to patch clamp. Assays of the invention also allow for less specific cytotoxicity to be measured on beating or bursting cells through the ability to monitor changes in cell adhesion and morphology in addition to the beating or bursting phenotype.

Burst or spike periods of neuronal cells can be detected with the methods of the invention. Input-driven or intrinsic bursting of neurons can be determined. Specific patterns that can be detected include, for example, tonic or regular spiking by neurons that are constantly active (e.g., interneurons), phasic bursting by neurons that fire in bursts, and fast spiking by neurons with high firing rates (e.g., cortical inhibitory interneurons, cells of the globus pallidus, retinal ganglion cells). Therefore, the rate, frequency, and kinetic profile of neuronal cells bursting or spiking in culture can be determined with the assays of the invention. Furthermore, the effect of compounds on these burst or spike patterns can be detected with methods of the invention.

The rate, frequency and kinetic profile can be detected in real time using a high speed, high resolution instrument, such as the BIND® READER (i.e., a colorimetric resonant reflectance biosensor system), and corresponding algorithms to quantify data. See, e.g., U.S. Pat. Nos. 7,422,891; 7,327,454, 7,301,628, 7,292,336; 7,170,599; 7,158,230; 7,142,296; 7,118,710. Additionally, cells and their differential morphological and adhesional responses to stimuli can be detected in real time with these methods.

The invention provides methods for screening a compound for an effect on cells, cell aggregates or tissues. For example, the rate, frequency, kinetic profile, or a combination thereof can be determined for any kind of cells exposed to any type of compound, compounds, environmental condition, environmental conditions, or combinations thereof. Cells, cell aggregates, or tissue are applied to the surface of a colorimetric resonant reflectance biosensor surface and one or more test compounds or environmental conditions are added to the cells, cell aggregates, or tissue. The compounds or environmental conditions can be added to the cells, cell aggregates or tissue prior to the cells, cell aggregates or tissue being applied to the biosensor surface. The PWV (or effective refractive index) of the cells, cell aggregates, or tissue is monitored over time. The PWV can be monitored before the compound or environmental conditions is added, while the compound or environmental condition is being added, after the compound or environmental condition is added and any combination thereof. A PWV reading (or other reading) can be taken about 1, 2, 3, 4, 5, 10, 20 or more times a second (or any range between about 1 and 20 times a second). A PWV reading can be taken about every 2, 5, 10, 20, 30, 45 or 60 seconds (or any range between about 2 and 60 seconds). A PWV reading can be taken about every 1, 2, 3, 4, 5, 10, 20, or 60 minutes (or any range between about 1 and 60 minutes).

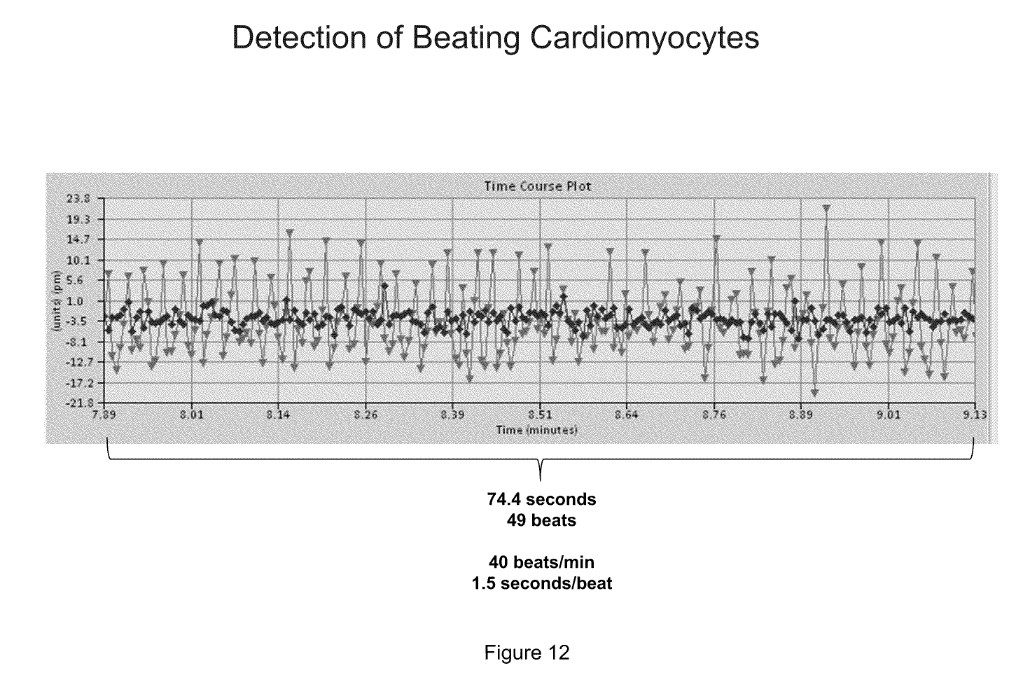

### Frequency

Where the cells are cardiomyocytes, the cells will beat in culture and the beating cells can generate a PWV pattern (alternating positive-negative PWV shift) or effective refractive index pattern that reveals the beating rate or frequency of the cells. See FIG. 12. The length of time between each beat can be determined. The effect of a compound, extracellular matrix, or environmental condition (e.g., salt concentration, buffer type, media type, serum type, temperature, oxygen concentration) on the frequency of the beating of the cells can be determined.

Where the cells are neurons, the cells will burst/spike in culture and the and the bursting/spiking cells can generate a PWV pattern or effective refractive index pattern that reveals the bursting/spiking rate or frequency of the bursting/spiking. The length of time between each burst or spike can be determined. The effect of a compound, extracellular matrix, or environmental condition on the frequency of the bursting or spiking of the cells can be determined.

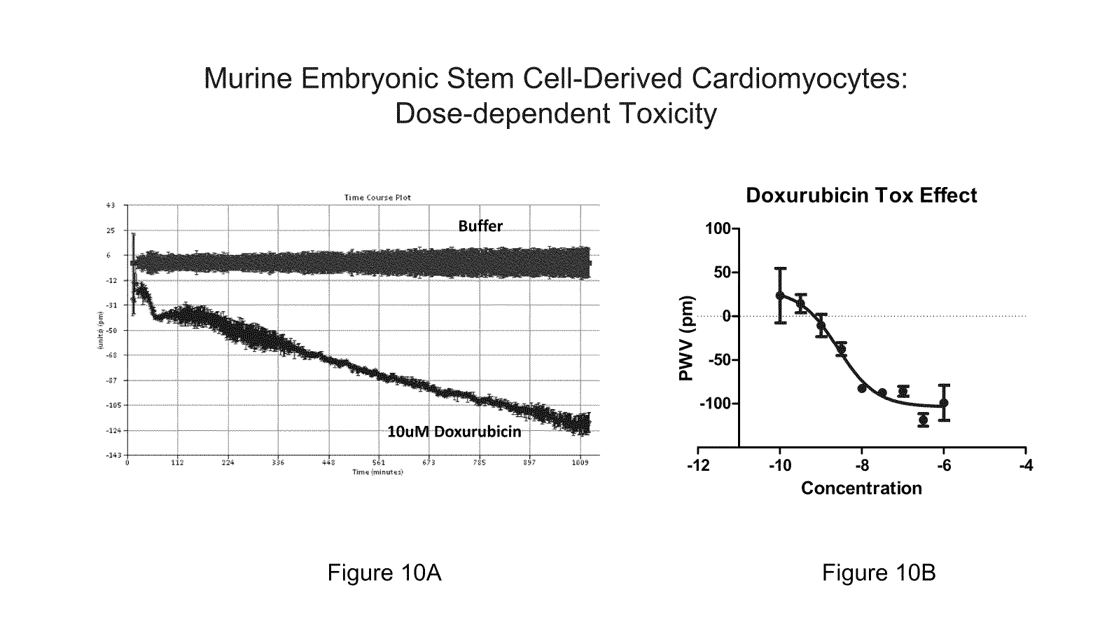

### Amplitude

Where the cells are cardiomyocytes, the cells can generate a PWV pattern or effective refractive index pattern that reveals the strength of the cardiomyocyte beating. This is an amplitude reading. In FIG. 10A-B, cardiomyocytes are treated with either buffer or doxorubicin. Where the cardiomyocytes are treated with buffer, the amplitude of the beating becomes stronger over time. That is, there is Y axis spread of the PWV reading grows larger over time as the cells beat stronger in culture. In some cases, for example, a toxin might cause a decrease the amplitude of the PWV reading over time, while a buffer or beneficial compound might retain or increase the amplitude of the PWV reading over time.

Where the cells are neurons, the cells can generate a PWV pattern or effective refractive index pattern that reveals the strength of the bursts or spikes. This is an amplitude reading.

### Kinetic Profile

The kinetic profile is a collection of about 2, 5, 10, 20, 50, 100, 250, 500, 1,000 or more PWVs (or effective index values) of a cell population taken over time (about 1, 5, 10, 30, 60 seconds, about 1, 2, 3, 4, 5, 10, 20, 40, 60 or more minutes). The kinetic profile reveals changes in PWV over time and represents a unique signature of the test compound or environmental condition. For example, where the test compound is a toxin, the PWVs may decline over time as the cells become weaker and then die. Where the compound is neutral or provides a benefit to the cells the PWV over time may increase, indicating a strengthening or growing of the cells. A kinetic profile can also be PWVs of a cell population taken for two or more differing concentrations of a test compound. The kinetic profile reveals changes in PWV over differing concentrations and represents a unique signature of the test compound or environmental condition.

### Cells

The assays of the invention can be used with any cells including, for example human or mammalian embryonic or human or mammalian adult stem cells and induced pluripotent stem cells. Induced pluripotent stem cells are pluripotent stem cells that are artificially produced from non-pluripotent cells, such as adult somatic cells, by inducing forced expression of certain genes. The induced pluripotent stem cells can be, for example, neurons, neural stem cells, cardiomyocytes, teratomas, or embryoid bodies. Other cells that can be used include, for example, cardiomyocytes, hepatocytes, neurons or combinations thereof including, for example, combinations or mixtures of hepatocytes and cardiomyocytes. A neuron can be any type of neuron, including, for example, type I neurons, type II neurons, interneurons, basket cells, Betz cells, medium spiny neurons, Purkinje cells, pyramidal cells, Renshaw cells, granule cells, anterior horn cells, or motorneurons.

### Methods of Screening Cells

One embodiment of the invention provides methods for screening a compound or environmental condition for an effect on cells, cell aggregates, or tissue. Cells, cell aggregates, or tissue are applied to a colorimetric resonant reflectance biosensor (or other biosensor) surface. The cells, cell aggregates, or tissue are contacted with a test compound or environmental condition. Periodic or continuous peak wavelength values are determined and recorded during a time course of the assay. The peak wavelength values are analyzed for frequency, amplitude, or kinetic profile or a combination thereof over the time course of the assay. A change in frequency, amplitude, or kinetic profile after the compound or environmental condition is contacted with the cells, cell aggregates, or tissue indicates that the compound or environmental condition has an effect on the cells, cell aggregates, or tissue. Two or more concentrations of the compound can be added to one or more populations the cells, cell aggregates, or tissue at one or more distinct locations on the biosensor surface. Where the one or more cell, cell aggregate or tissue populations comprise two or more populations (e.g., 2, 3, 4, 5, 10, 15, 20, 100, 250, or more) the populations may be the same or different.

A decreased frequency over the time course of the assay can indicate a negative effect of the compound or environmental condition on the cells and a decreased amplitude over the time course of the assay can indicate a negative effect of the compound or environmental condition on the cells. A negative effect can be a weakening of the cells or death of the cells. A decreased frequency with increasing compound concentration can indicate a negative effect of the compound on the cells and a decreased amplitude with increasing compound concentration can indicate a negative effect of the compound or environmental condition on the cells.

An increased frequency over the time course of the assay can indicate a neutral or positive effect of the compound or environmental condition on the cells and an increase in amplitude over the time course of the assay can indicate a neutral or positive effect of the compound or environmental condition on the cells. A positive effect or a neutral effect can be cells strengthening, growing, or multiplying. An increase in frequency with increasing compound concentration can indicate a neutral or positive effect of the compound on the cells and an increase in amplitude with increasing compound concentration can indicate a neutral or positive effect of the compound or environmental condition on the cells.

The peak wavelength values can be analyzed for kinetic profile, wherein a kinetic profile that moves from a positive peak wavelength value to a negative peak wavelength value over the time course of the assay can indicate a negative effect of the compound or environmental condition on the cells. A kinetic profile that moves from a positive peak wavelength value to a negative peak wavelength value with increasing concentration of the compound can indicate a negative effect of the compound or environmental condition on the cells.

The peak wavelength values can be analyzed for kinetic profile, wherein a kinetic profile that moves from a negative or neutral peak wavelength value to a neutral or positive peak wavelength value over the time course of the assay can indicate a positive or neutral effect of the compound or environmental condition on the cells. A kinetic profile that moves from a negative or neutral peak wavelength value to a positive or neutral peak wavelength value with increasing concentration of the compound can indicate a positive of neutral effect of the compound or environmental condition on the cells.

The cells can be human or mammalian stem cells, human or mammalian induced pluripotent stem cells (such as cardiomyocytes), cells differentiated from the human or mammalian induced pluripotent cells (such as cardiomyocytes), neural stem cells, neurons, cardiomyocyte stem cells, cardiomyocytes, myocardiocyteal muscle cells, hepatic stem cells, hepatocytes or combinations thereof, such as combinations or mixtures of cardiomyocytes and hepatocytes. Cell aggregates can be any type of cell aggregates, e.g., embroid bodies. Tissues can be any type of tissue, e.g., liver tissue, cardiac tissue, brain tissue, neuronal tissue, or spinal cord tissue.

The compound can be, e.g., a drug, a calcium channel blocker, a β-adrenoreceptor agonist, an α-adrenoreceptor agonist, any test reagent, a polypeptide, a polynucleotide, a modifier of a hERG channel, or a toxin.

### Methods for Reducing Risk of Drug Toxicity

One embodiment of the invention provides a method for reducing the risk of drug toxicity in a subject, such as a human or mammalian subject. One or more cells differentiated from an induced pluripotent stem cell line generated from the subject can be contacted with a dose of a pharmacological agent. The contacted one or more cells are assayed for toxicity. The cells are applied to a colorimetric resonant reflectance biosensor (or other biosensor) surface. The cells are contacted with the pharmacological agent and periodic peak wavelength values are detected during a time course of the assay. The peak wavelength values are analyzed for frequency, amplitude, or kinetic profile or a combination thereof over the time course of the assay. A negative change in frequency, amplitude, or kinetic profile after the pharmacological agent is contacted with the cells can indicate that the pharmacological agent has a negative toxicity effect on the cells. The pharmacological agent is prescribed or administered to the subject only if the pharmacological agent does not have a negative toxicity effect on the contacted cells.

Another embodiment of the invention provides a method for reducing the risk of drug toxicity in a subject, such as a human or mammalian subject. The method comprises contacting one or more cell populations differentiated from an induced pluripotent stem cell line generated from the subject with two or more dose concentrations of a pharmacological agent and assaying the contacted one or more cell populations for toxicity. The assaying comprises applying the one or more cell populations to a colorimetric resonant reflectance biosensor surface and contacting the one or more cell populations with two of more concentrations the pharmacological agent. One or more peak wavelength values are detected for each concentration of the pharmacological agent. The peak wavelength values are analyzed for frequency, amplitude, or kinetic profile or a combination thereof for each concentration of the pharmacological agent. A negative change in frequency, amplitude, or kinetic profile after the pharmacological agent is contacted with the cells can indicate that the pharmacological agent concentration has a negative toxicity effect on the cells. The pharmacological agent is prescribed or administered to the subject only if the pharmacological agent concentration does not have a negative toxicity effect in the contacted cells.

### Methods for Screening a Compound for Neutralizing Activity

One embodiment of the invention provides methods for screening a compound for neutralizing activity on a known toxin or negative environmental condition (i.e., any environment condition that weakens or kills the cells or has a negative impact on cell growth or cell multiplication). The method comprises applying cells, cell aggregates, or tissue to a colorimetric resonant reflectance biosensor (or other biosensor) surface and contacting the cells, cell aggregates, or tissue with the known toxin and a compound. Periodic or continuous peak wavelength values are detected during a time course of the assay. The peak wavelength values are analyzed for frequency, amplitude, or kinetic profile or a combination thereof over the time course of the assay. A positive change in frequency, amplitude, or kinetic profile after the compound is contacted with the cells, cell aggregates, or tissue can indicate that the compound has a neutralizing effect on the toxin.

Another embodiment of the invention provides methods of screening a compound for neutralizing activity on a known toxin. The method comprises applying one or more cells, cell aggregates, or tissue populations to a colorimetric resonant reflectance biosensor (or other biosensor) surface and contacting the one or more cells, cell aggregates, or tissue populations with the known toxin and a compound at two or more compound concentrations. Periodic or continuous peak wavelength values are detected during a time course of the assay for each compound concentration. Peak wavelength values are analyzed for frequency, amplitude, or kinetic profile or a combination thereof for each compound concentration over the time course of the assay. A positive change in frequency, amplitude, or kinetic profile after the compound is contacted with the cells, cell aggregates, or tissue can indicate that the compound has a neutralizing effect on the toxin. The contacting step can further include contacting the cells, cell aggregates, or tissue with at least one second compound (e.g., 1, 2, 3, 4, 5, 10, 20, 30, 40, 50 or more) or at least one second environmental condition (e.g., 1, 2, 3, 4, 5, 10, 20, 30, 40, 50 or more) in the presence of the first compound or environmental condition.

### Methods of Screening Compounds for Signature Kinetic Profiles

An embodiment of the invention provides a method of screening a test toxin or compound for a signature kinetic profile to determine a class or subclass of the toxin or compound. Cells, cell aggregates, or tissue are applied to a colorimetric resonant reflectance biosensor (or other biosensor) surface and the cells, cell aggregates, or tissue are contacted with the test toxin or compound. Periodic or continuous peak wavelength values are determined during a time course of the assay. The peak wavelength values are analyzed for frequency, amplitude, or kinetic profile or a combination thereof over the time course of the assay to generate a signature kinetic profile of the effects of the test toxin or test compound on the cells, cell aggregates, or tissue. The signature kinetic profile of the test toxin or compound is compared to signature kinetic profiles of known toxins or compounds, wherein the test toxin or compound is placed into a class or subclass of toxins or compounds having a similar signature kinetic profile as the test toxin or test compound.

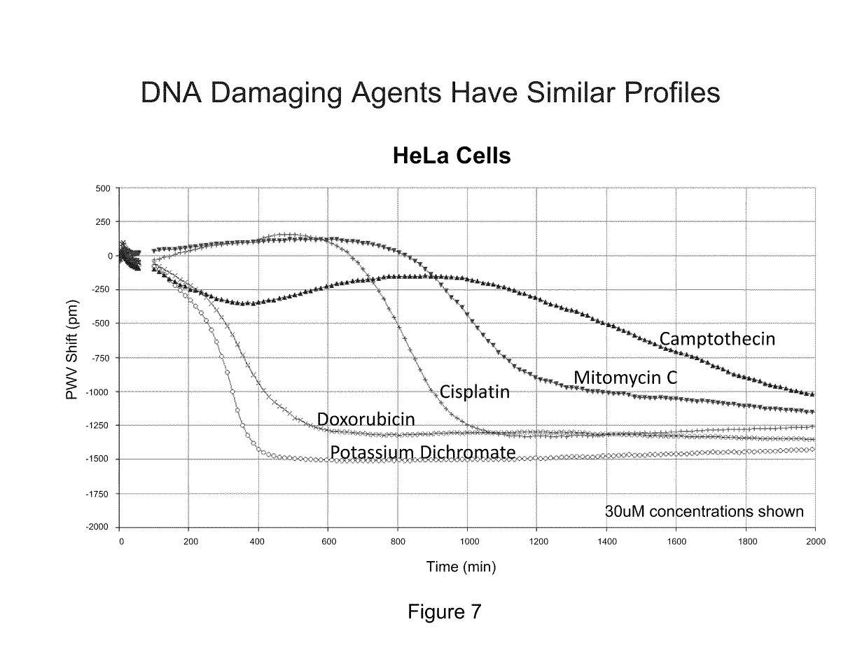

Signature kinetic profiles are obtained by determining kinetic profiles for two or more (e.g., 2, 3, 4, 5, 10, 15, 20, or more) classes or subclasses of toxins (e.g., DNA damaging agents, topoisomerase inhibitors, DNA gyrase inhibitors, RNA inhibitors, ion channel inhibitors, etc.) or compounds. Where the two or more kinetic profiles for toxins or compounds in the same class or subclass are similar (see, e.g., FIG. 7), then the combination of kinetic profile is a signature kinetic profile.

### Methods of Screening for Effects on Sinus Rhythm of Cardiomyocytes

The invention provides methods for determining the effect of a test compound or environmental condition on the sinus rhythm of cardiomyocytes. The method compromises applying any type of cardiomyocytes to a colorimetric resonant reflectance biosensor surface, a dielectric film stack biosensor surface, or a grating-based waveguide biosensor surface. The cardiomyocytes are contacted with the compound or environmental condition and periodic or continuous peak wavelength values or effective refractive index values are detected during a time course of the assay. The peak wavelength values or effective refractive index values are analyzed for sinus rhythm over the time course of the assay. A change in the sinus rhythm after the compound or environmental condition is contacted with the cardiomyocytes indicates that the compound or environmental condition has an effect on the sinus rhythm of the cardiomyocytes.

Very specific effects of compounds, combination of compounds, or environmental conditions on sinus rhythm waves, segments and intervals can be determined. For example, the prolongation (or shortening) of the QT interval can be determined using methods of the invention. The heart rate corrected QT interval, QTc, can also be determined using Bazett's formula. Changes in length (longer or shorter) of the PR interval, PR segment, ST segment can be determined. Additionally, widening of the QRS complex, P wave, Q wave, R wave, S wave or T wave; abnormal deflections of the QRS complex, P wave, Q wave, R wave, S wave or T wave; duration of the QRS complex, P wave, Q wave, R wave, S wave or T wave; amplitude of the QRS complex, P wave, Q wave, R wave, S wave or T wave; and morphology of the QRS complex, P wave, Q wave, R wave, S wave or T wave (e.g., a notch in the T wave) can be detected by the methods of the invention.

All patents, patent applications, and other scientific or technical writings referred to anywhere herein are incorporated by reference in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

### EXAMPLES

### Example 1

### Signature Kinetic Profiles

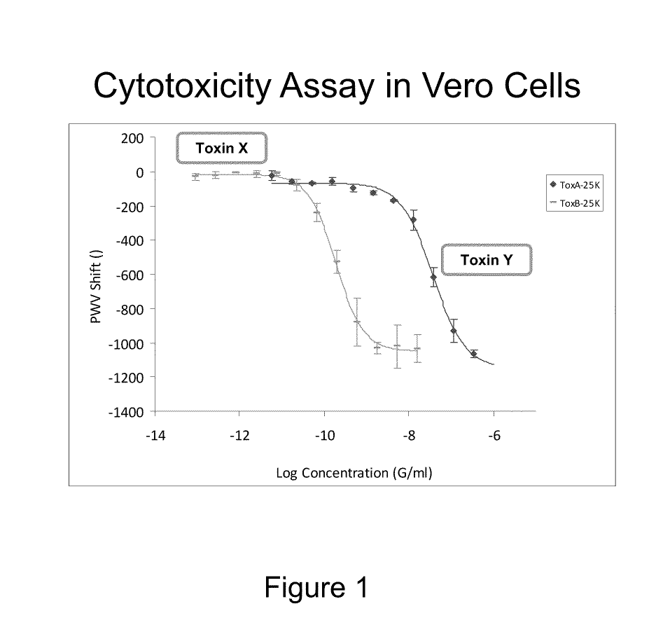

Vero cells were plated in complete media a 25,000 cells/well of a colorimetric resonant reflectance biosensor microtiter plate. The cells were exposed to one of two toxins at several different concentrations for 16 hours. The toxic effect of the toxins was evident at between 1.5 and 2.0 hours after toxin addition. The IC₅₀ for Toxin X was 37 ng/ml. The IC₅₀ for Toxin Y was 0.187 ng/ml. The kinetic profile of Toxin X and Toxin Y is shown in FIG. 1. There is a concentration dependant negative shift in PWV as the toxins kill the cells.

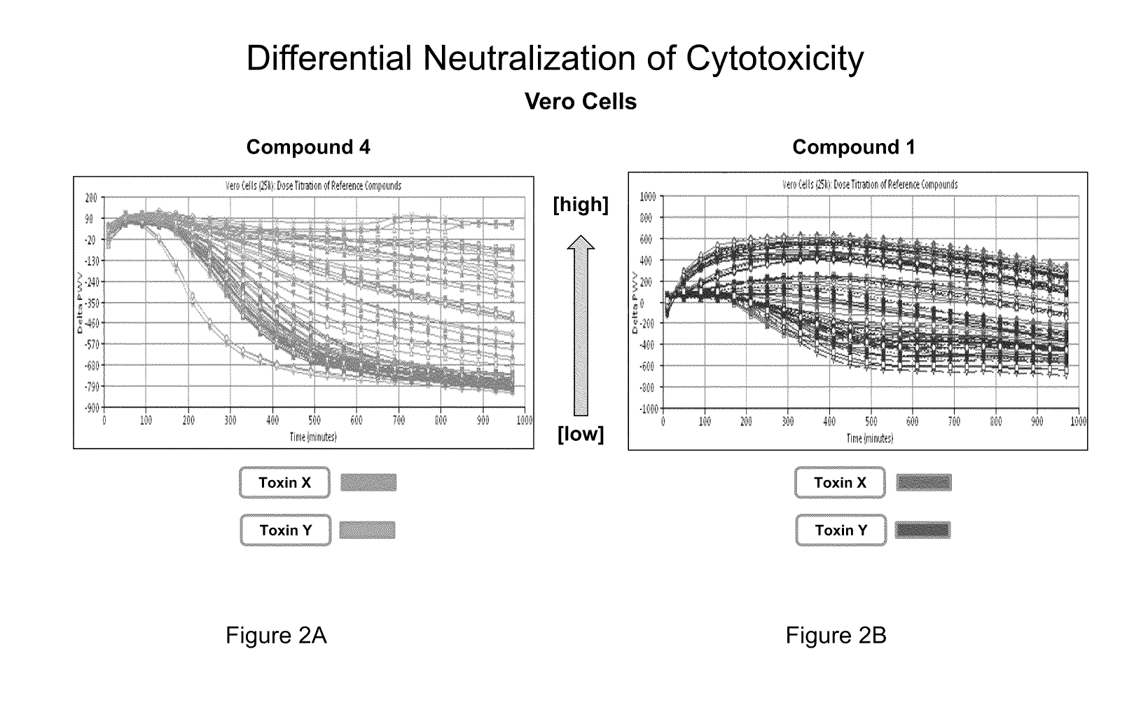

In another experiment, Vero cells were again plated into wells of a colorimetric resonant reflectance biosensor microtiter plate. Two toxins were added to the cells, Toxin X or Toxin Y. Compound 4 or Compound 1 were also added to the wells. Compound 4 blocks Toxin X, but does not block Toxin Y. Compound I blocks both Toxin X and Toxin Y. In wells where Compound 4 was added Toxin X was blocked. FIG. 2A shows the blocking of cell death by Toxin X over time (lighter lines). Cell death caused by Toxin Y was not blocked as demonstrated by a shift to negative PWVs. See FIG. 2A, darker lines. Where Compound 1 was added to the cells, cell death by the toxins was blocked by Compound 1 and a neutralization of negative PWVs is seen. See FIG. 2B. Therefore, assays of the invention can be used to screen for compounds that neutralize toxins.

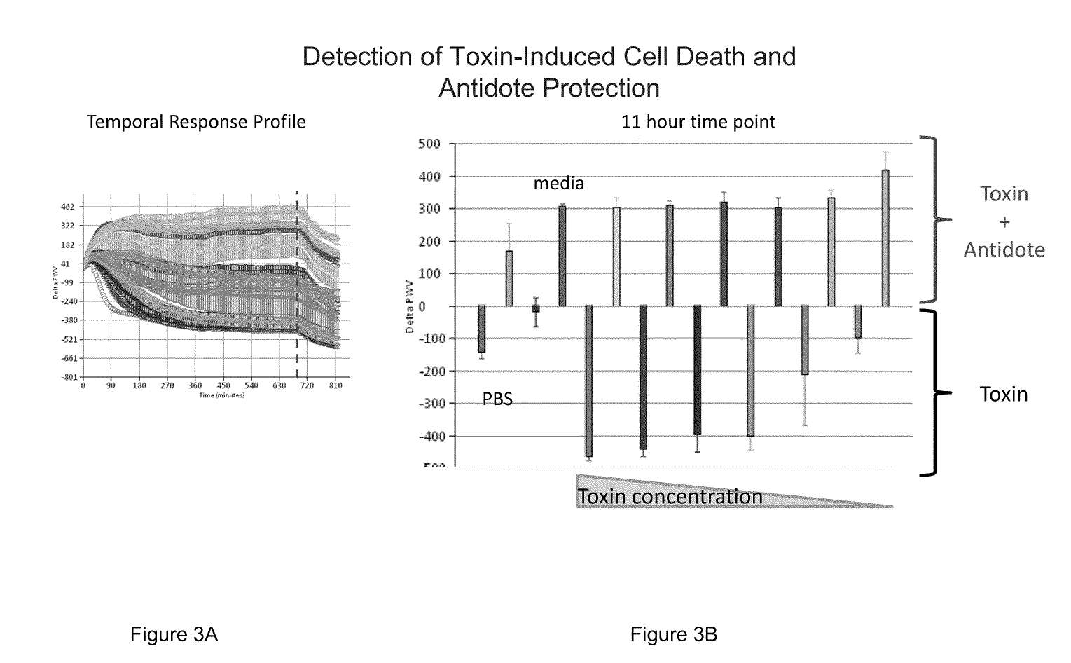

FIG. 3A-B demonstrates an experiment where increasing concentrations of toxin were mixed with neutralizing doses of an antidote. CHO cells were plated at 25,000 cells/well on a CA2 384-well BIND™ biosensor plate in complete media for 3-4 hours. A toxin or a toxin:antidote mixture was then added to the wells. Cells were monitored using a BIND™ biosensor plate reader for 15-16 hours at room temperature. The results are shown in FIG. 3A-B. FIG. 3A shows the temporal response profile and FIG. 3B shows the results at an 11 hour time point. The antidote protects cells from cell death as indicated by neutralization of the dose-dependent, negative PWV shift elicited by toxin.

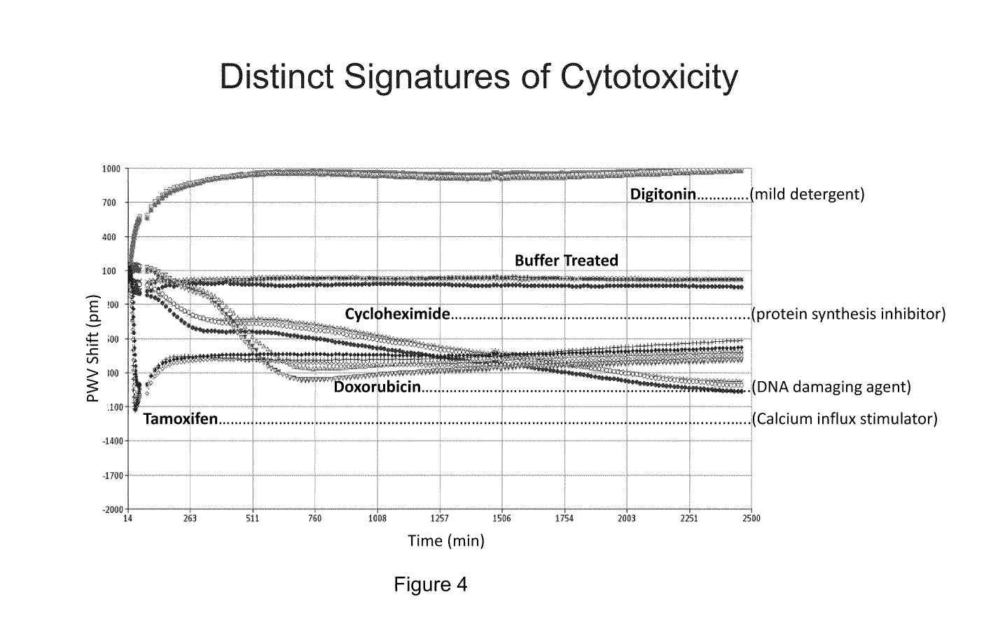

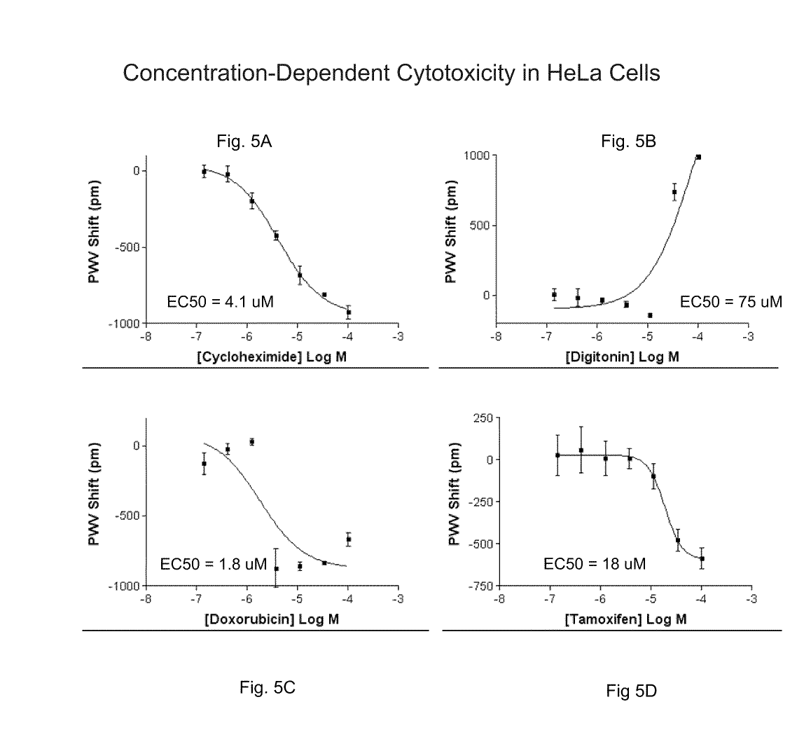

In another experiment HeLa cells were treated with 100 uM of tamoxifen (a calcium influx stimulator), doxorubicin (a DNA damaging agent), cycloheximide (a protein synthesis inhibitor), digitonin (a mild detergent), and a buffer, and monitored every 15 minutes on a BIND™ Reader for 40 hours in a 37° C. incubator. The cytotoxic compounds each have distinct mechanisms of action that have distinct kinetic profiles in the assays of the invention. See FIG. 4. Toxins can be tested for their effect on differing types of cells and can be placed into a class or sub-class of toxins (e.g. calcium influx stimulator) based on their kinetic profiles. These assays have a higher throughput than electrical impedance testing for screening and profiling of toxic compounds. FIG. 5 shows the PWV shift for each of the cytotoxic agents in relation to the concentration of the cytotoxic agent (FIG. 5A: cycloheximide; FIG. 5B: digitonin; FIG. 5C: doxorubicin; FIG. 5D: tamoxifen).

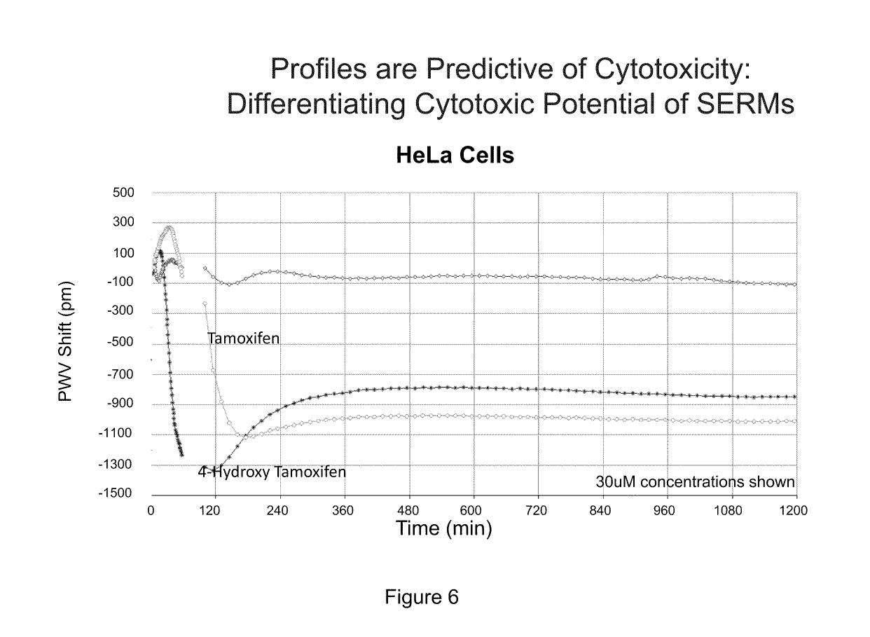

The cytotoxic activity of tamoxifen is thought to occur by inducing calcium mobilization. The kinetic profile of tamoxifen includes a sharp downward shift in PWV at early time points. See FIG. 6. 4-hydroxy-tamoxifen is a metabolite of tamoxifen, with higher affinity for estrogen receptor and greater toxicity. 4-hydroxy-tamoxifen has a kinetic profile that has a faster onset of toxicity than tamoxifen. See FIG. 6. Raloxifene (unlabeled line) is from the same class (SERM, or estrogen receptor modulators) as tamoxifen, but has dramatically reduced side effects and reduced cytotoxicity. The kinetic profile of raloxifene demonstrates lack of cytotoxic response. See FIG. 6.

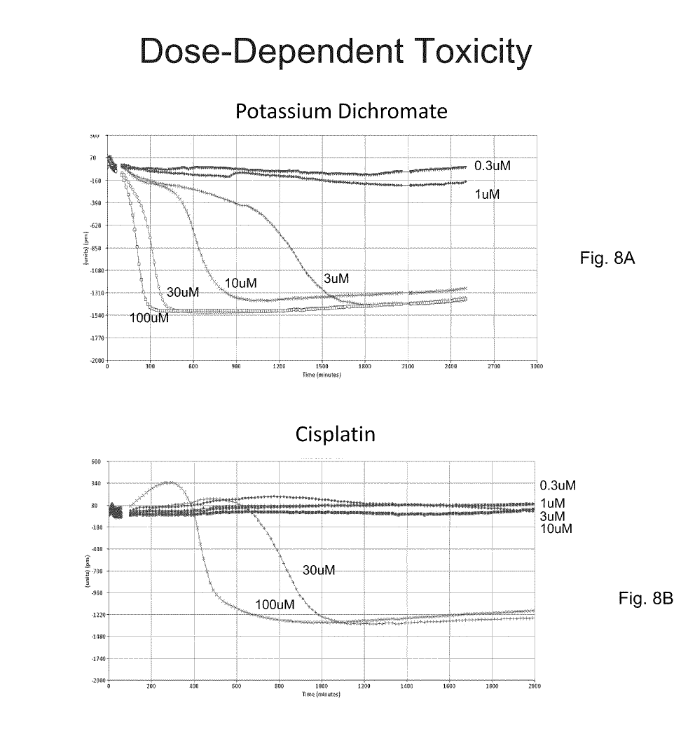

FIG. 7 shows the PWVs for several known DNA damaging agents over time. All have same basic profile of a sudden onset, steep decline, followed by a negative PWV plateau. Cisplatin (intercalating agent), potassium dichromate (intercalating agent), doxorubicin (crosslinking agent), and mitomycin (crosslinking agent) all have similar profiles and all are direct DNA damaging agents. Camptothecin is a topoisomerase inhibitor and has somewhat different kinetic profile. FIG. 8A-B shows the PWVs for differing concentrations of potassium dichromate (FIG. 8A) and cisplatin (FIG. 8B).

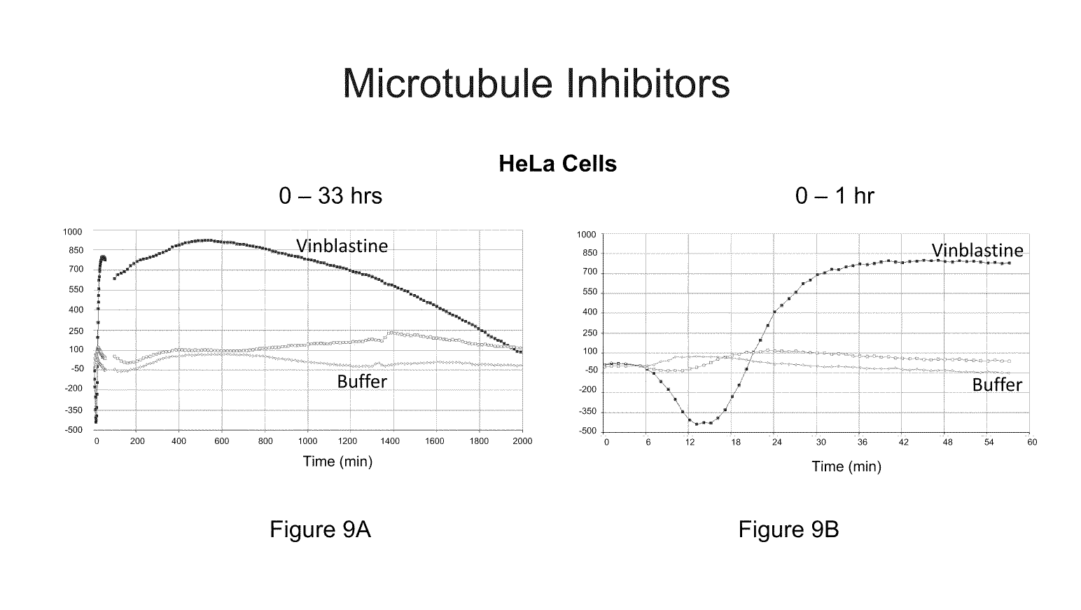

Many compounds have undesired effects on microtubules. Vinblastine binds to tubulin and disrupts microtubule formation. The kinetic profile of vinblastine is distinct from other toxins. See FIG. 9A-B. The earlier time points (FIG. 9B) show a rapid acute response and then a gradual negative PWV shift over the long term. The early response is likely a result of acute morphological effects elicited by microtubule disruption. The longer-term negative PWV response is likely the result of cell death (FIG. 9A). This assay highlights the potential of multiple readouts (on target+toxicity) in one assay.

### Example 2

### Frequency and Rate Determinations

mES-derived cardiomyocytes (Cor.At) were obtained from Axiogenesis/Lonza. 5,000 cells per well were plated on fibronectin-coated 384 well biosensor plates for 24 h before experiment. These cells beat in culture. Cells were treated with doxorubicin for 17 h at 37° C. with constant monitoring using the BIND™ Reader. In FIG. 10A the buffer reading demonstrates the beating of the cells in culture. There is an oscillation of PWVs that indicates the beating of the cells. The amplitude of the beating becomes stronger over time. That is, there is Y axis spread of the PWV reading grows larger over time as the cells beat stronger in culture. Where doxorubicin is added to the cells, the PWV readings become negative. Additionally, the amplitude of the cells becomes weaker over time. The effect of doxorubicin is dose dependant. See FIG. 10B.

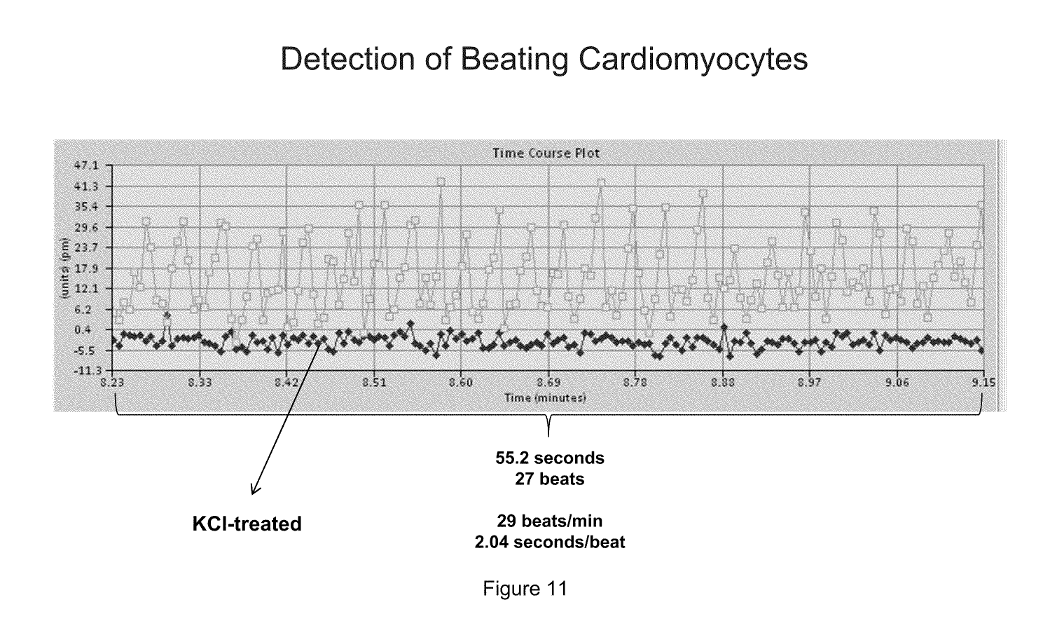

Murine embryonic stem cell-derived cardiomyocytes were added to wells of a biosensor and a BIND™ Reader recorded PWVs at the rate of 4 reads per second. One well was treated with KCl and another well was not treated. The results are shown in FIG. 11. The cells beat synchronously when cultured on BIND™ optical biosensors and the frequency of the beating can be detected. See FIG. 12. KCl treatment leads to a loss of amplitude of the beating and decrease in the frequency of the beating. The BIND™ Reader detects beating frequency and rate as oscillations of positive-negative PWV shifts. Kinetic PWV profiles can be used to accurately measure beating rate and frequency. Therefore, the assay provides an ultra high-throughput assay to measure off-target drug effects on cardiotoxicity and contractility.

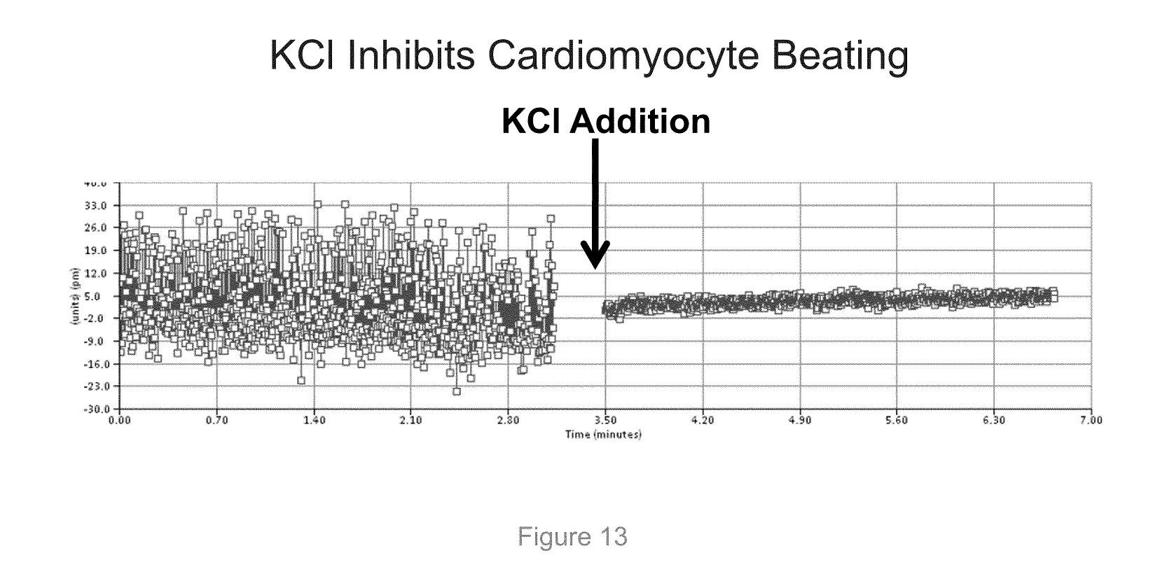

In another experiment the cardiomyocytes were plated at 20,000 cells per well on fibronectin coated biosensor plates. The cells were incubated for 72 h at 37° C. before the experiment. The cells were monitored for 3 minutes before KCl was added to 50 mM final concentration. The results are shown in FIG. 13. The rate and frequency of the beating of the cells were drastically reduced upon addition of the KCl to wells. This demonstrates that the monitoring of cells before compound addition and after compound addition can be done in a single well.

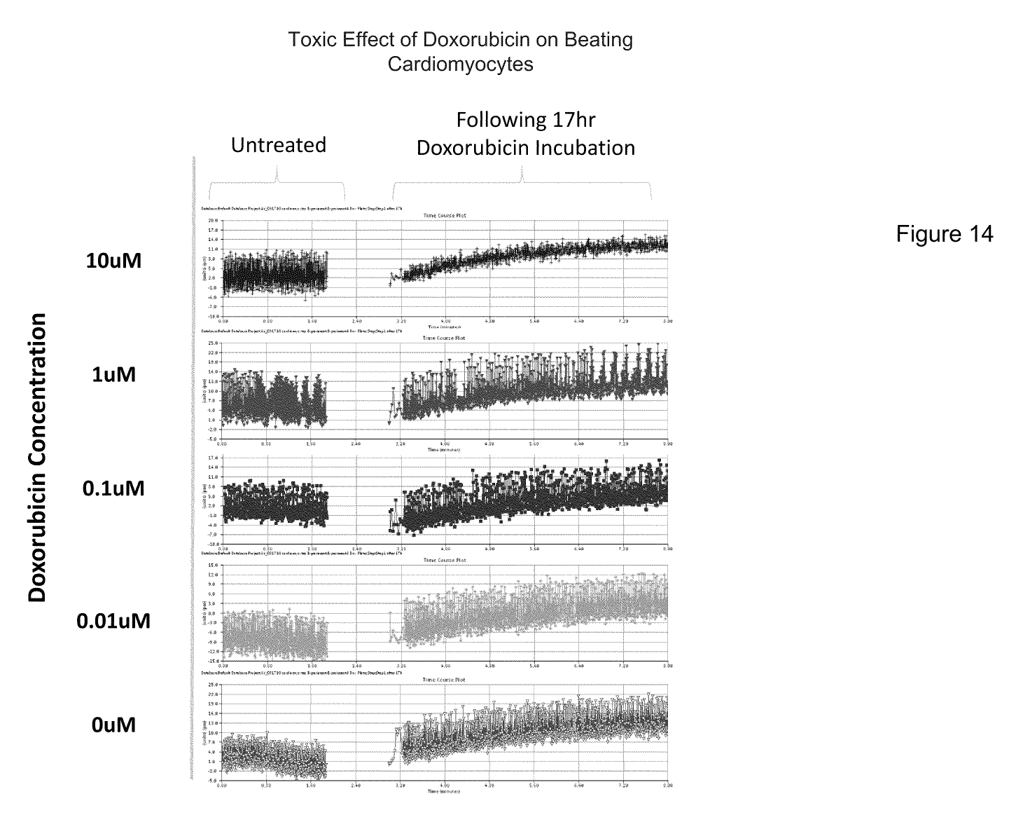

In another experiment cardiomyocytes were treated for 17 hours with several different concentrations of doxorubicin (10 μM, 1 μM, 0.1 μM, 0.01 μM, and 0 μM). The beating amplitude and frequency was measured at 4 reads/second prior to doxorubicin treatment, then again after the 17 hour doxorubicin incubation. The results are shown in FIG. 14. The amplitude and frequency of beating is disrupted by doxorubicin in a concentration-dependent manner.

### Example 3

An Ocean Optics HT2000+ spectrometer in a BIND™ Cartridge Reader was altered so that the slit that allows light into the spectrometer was widened, allowing more light to enter the enter the spectrometer. Therefore, recordings of up to 1000 Hz, i.e. up to 1000 readings/second can be obtained when the BIND™ Cartridge Reader is “parked” (i.e., the BIND™ Cartridge Reader remains in one position over, e.g., a well holding cells). The recordings are made continually in real-time. Recordings can be taken at about 2, 4, 10, 50, 80, 100, 250, 280, 300, 400, 500, 600, 700, 800, 900, 1,000 or more Hz (or any range between about 2 and about 1,000 Hz).

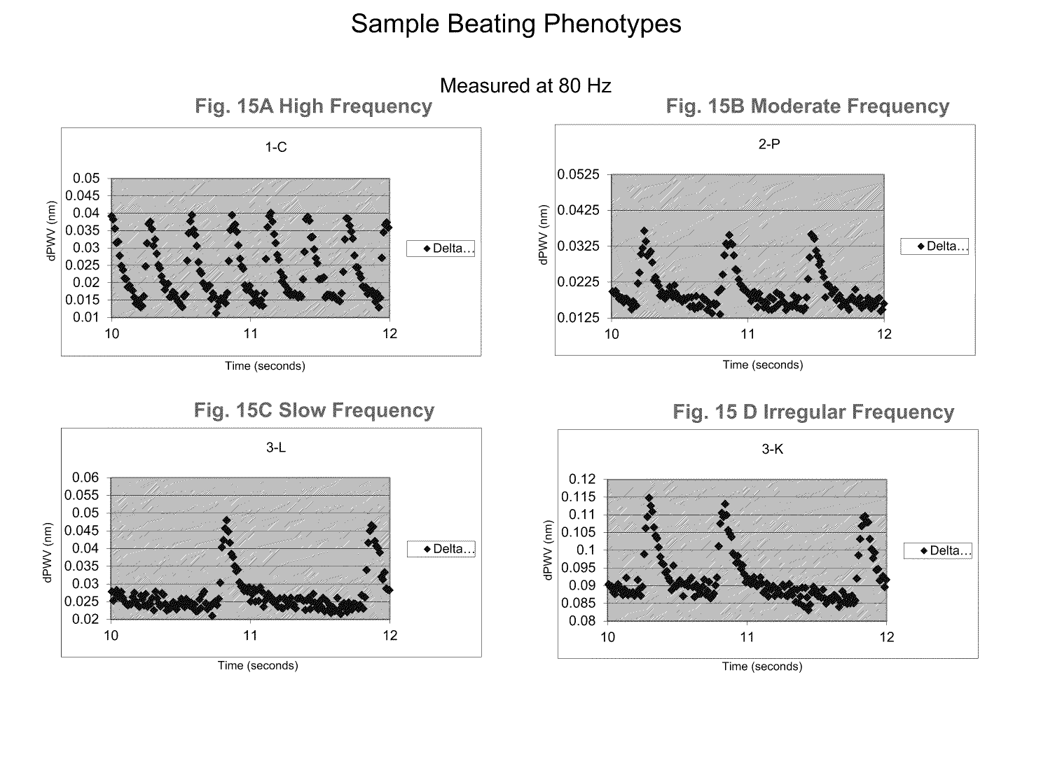

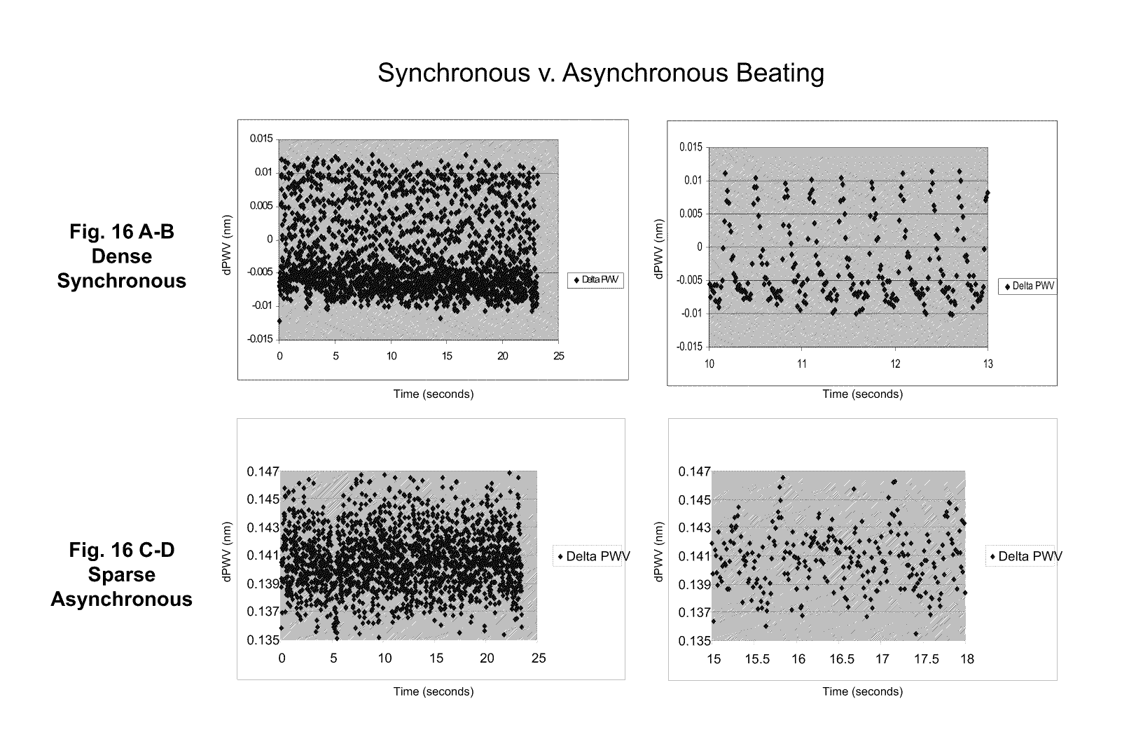

The refined BIND™ Cartridge Reader was used to take measurements of cardiomyocyte beating. FIG. 15 shows readings taken at 80 Hz. By comparison, the data in Examples 1 and 2 was measured at 4 Hz. The increased sampling rate allows a much more refined look at the shape of each individual beat. Different beating “phenotypes” can be determined from well-to-well, in addition, the difference between synchronous beating across the well and asynchronous beating can be determined. FIG. 15A shows high frequency beating, FIG. 15B shows moderate frequency beating, FIG. 15C shows slow frequency beating, FIG. 15D shows irregular frequency beating. FIG. 16A-16B shows dense synchronous beating. FIG. 16C-D shows sparse asynchronous beating. The effects of compounds that have cardiotoxic properties, such as blocking hERG channels and/or affect QT prolongation can be examined. The QT interval is a measure of the time between the start of the Q wave and the end of the T wave in the heart's electrical cycle. A prolonged QT interval is a risk factor for ventricular tachyarrhythmias and sudden death. Methods of the invention can be used to screen for compounds that result in QT prolongation in moderate/high throughput screens. The methods of the invention can measure subtle-to-significant effects on the beat of cardiomyocytes in culture, which are predictive of effects of beating hearts in vivo.

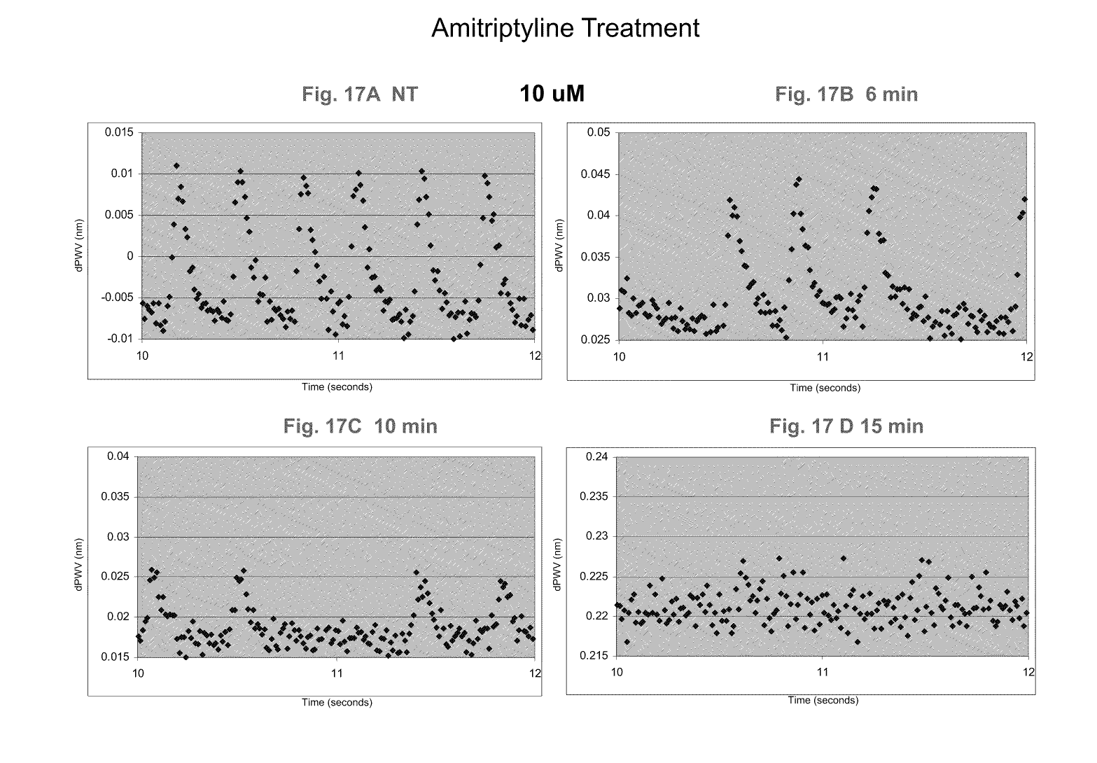

Amitriptyline is a tricyclic antidepressant sold under the trade name Elavil™. Amitriptyline functions primarily as a serotonin-norepinephrine reuptake inhibitor by modulating transporters for both transmitters. It is associated with heart arrhythmias due to hERG channel modulation and QT prolongation. Amitriptyline was added to cardiomyocytes that were beating in culture and PWVs were constantly monitored. FIG. 17A shows the PWVs of the cells over time prior to the addition of the amitriptyline. FIGS. 17B, 17C, and 17D show the PWVs of the cardiomyocytes over time at 6 minutes, 10 minutes, and 15 minutes, respectively, after the addition of amitriptyline. The change from regular, synchronous beating to irregular, non-synchronous beating can clearly be seen after the addition of the amitriptyline.

Therefore, methods of the invention can be used to screen the effect of compounds, a combination of compounds, or environmental condition on the kinetic profile, beating frequency and beating rate of cardiomyocytes and other cells.

We claim:

1. A method for screening a compound or environmental condition for an effect on cells, cell aggregates, or tissue comprising:
(a) applying the cells, cell aggregates, or tissue to a colorimetric resonant reflectance biosensor surface, a dielectric film stack biosensor surface, or a grating-based waveguide biosensor surface;
(b) contacting the cells, cell aggregates, or tissue with the compound or environmental condition;
(c) detecting periodic or continuous peak wavelength values or effective refractive index values during a time course;
(d) analyzing the peak wavelength values or effective refractive index values for frequency, amplitude, or kinetic profile, or a combination thereof over the time course; wherein a change in frequency, amplitude, or kinetic profile after the compound or environmental condition is contacted with the cells, cell aggregates, or tissue indicates that the compound or environmental condition has an effect on the cells, cell aggregates, or tissue.

2. The method of claim 1, wherein two or more concentrations of the compound are added to one or more populations the cells, cell aggregates, or tissue at two or more distinct locations on the biosensor surface.

3. The method of claim 1, wherein the cells are stem cells, human or mammalian induced pluripotent stem cells, cells differentiated from the human or mammalian induced pluripotent cells, neural stem cells, neurons, cardiomyocyte stem cells, cardiomyocytes, hepatic stem cells, hepatocytes. or combinations thereof.

4. The method of claim 3, wherein the human or mammalian induced pluripotent stem cell line, or cells differentiated from the human or mammalian induced pluripotent cells are cardiomyocytes.

5. The method of claim 1, wherein the peak wavelength values or effective refractive index values are analyzed for frequency or amplitude, wherein a decreased frequency over the time course of the assay indicates a negative effect of the compound or environmental condition on the cells, cell aggregates, or tissue, and wherein a decreased amplitude over the time course of the assay indicates a negative effect of the compound or environmental condition on the cells, cell aggregates or tissue.

6. The method of claim 2, wherein the peak wavelength values or effective refractive index values are analyzed for frequency or amplitude, wherein a decreased frequency with increasing compound concentration indicates a negative effect of the compound on the cells, cell aggregates, or tissue and wherein a decreased amplitude with increasing compound concentration indicates a negative effect of the compound or environmental condition on the cells, cell aggregates, or tissue.

7. The method of claim 1, wherein the peak wavelength values are analyzed for kinetic profile, wherein a kinetic profile that moves from a positive peak wavelength value to a negative peak wavelength value over the time course indicates a negative effect of the compound or environmental condition on the cells, cell aggregates, or tissue.

8. The method of claim 2, wherein the peak wavelength values are analyzed for kinetic profile, wherein a kinetic profile that moves from a positive peak wavelength value to a negative peak wavelength value with increasing concentration of the compound indicates a negative effect of the compound or environmental condition on the cells, cell aggregates, or tissue.

9. The method of claim 1, wherein the compound is a drug, a calcium channel blocker, a β-adrenoreceptor agonist, an α-adrenoreceptor agonist, test reagent, a polypeptide, a polynucleotide, a modifier of a hERG channel, or a toxin.

10. The method of claim 1, wherein the cell aggregates are embroid bodies.

11. A method for reducing the risk of pharmacological agent toxicity in a subject, comprising:
(a) contacting one or more cells differentiated from an induced pluripotent stem cell line generated from the subject with a dose of a pharmacological agent;
(b) assaying the contacted one or more cells for toxicity comprising:
(i) applying the cells to a colorimetric resonant reflectance biosensor surface, a dielectric film stack biosensor surface, or a grating-based waveguide biosensor surface;
(ii) contacting the cells with the pharmacological agent;
(iii) detecting periodic or continuous peak wavelength values or effective refractive index values during a time course;
(iv) analyzing the peak wavelength values or effective refractive index values for frequency, amplitude, or kinetic profile or a combination thereof over the time course; wherein a negative change in frequency, amplitude, or kinetic profile after the pharmacological agent is contacted with the cells indicates that the pharmacological agent has a negative toxicity effect on the cells;
(c) prescribing or administering the pharmacological agent to the subject only if the pharmacological agent does not have a negative toxicity effect on the contacted cells, thereby reducing the risk of pharmacological toxicity in a subject.

12. A method for reducing the risk of pharmacological agent toxicity in a subject, comprising:
(a) contacting one or more cell populations differentiated from an induced pluripotent stem cell line generated from the subject with two or more dose concentrations of a pharmacological agent;
(b) assaying the contacted one or more cell populations for toxicity comprising:
(i) applying the one or more cell populations to a colorimetric resonant reflectance biosensor surface, a dielectric film stack biosensor surface or a grating-based waveguide biosensor surface;
(ii) contacting the one or more cell populations with two of more concentrations the pharmacological agent;
(iii) detecting one or more peak wavelength values or effective refractive index values for each concentration of the pharmacological agent;
(iv) analyzing the peak wavelength values or effective refractive index values for frequency, amplitude, or kinetic profile or a combination thereof for each concentration of the pharmacological agent; wherein a negative change in frequency, amplitude, or kinetic profile after the pharmacological agent is contacted with the cells indicates that the pharmacological agent concentration has a negative toxicity effect on the cells;
(c) prescribing or administering the pharmacological agent to the subject only if the pharmacological agent concentration does not have a negative toxicity effect in the contacted cells, thereby reducing the risk of pharmacological toxicity in a subject.

13. A method of screening a compound for neutralizing activity on a toxin or negative environmental condition comprising:
(a) applying cells, cell aggregates, or tissue to a colorimetric resonant reflectance biosensor surface, a dielectric film stack biosensor surface, or a grating-based waveguide biosensor surface;
(b) contacting the cells, cell aggregates, or tissue with the toxin or negative environmental condition and the compound;
(c) detecting periodic or continuous peak wavelength values or effective refractive index values during a time course;
(d) analyzing peak wavelength values or effective refractive index values for frequency, amplitude, or kinetic profile or a combination thereof over the time course;
wherein a positive change in frequency, amplitude, or kinetic profile after the compound is contacted with the cells, cell aggregates, or tissue indicates that the compound has a neutralizing effect on the toxin or negative environmental condition.

14. A method of screening a compound for neutralizing activity on a toxin or negative environmental condition comprising:
(a) applying one or more cells, cell aggregates, or tissue populations to a colorimetric resonant reflectance biosensor surface, a dielectric film stack biosensor surface, or a grating-based waveguide biosensor surface;
(b) contacting the one or more cells, cell aggregates, or tissue populations with the toxin or negative environmental condition and the compound at two or more compound concentrations;
(c) detecting periodic or continuous peak wavelength values or effective refractive index values during a time course for each compound concentration;
(d) analyzing peak wavelength values or effective refractive index values for frequency, amplitude, or kinetic profile or a combination thereof for each compound concentration over the time course;
wherein a positive change in frequency, amplitude, or kinetic profile after the compound is contacted with the cells, cell aggregates, or tissue indicates that the compound has a neutralizing effect on the toxin or negative environmental condition.

15. The method of claim 1, wherein said contacting step further includes contacting the cells, cell aggregates, or tissue with at least one second compound or at least one second environmental condition in the presence of the first compound or the first environmental condition.

16. A method of screening a test toxin for a signature kinetic profile to determine a class or subclass of the test toxin comprising:
(a) applying cells, cell aggregates, or tissue to a colorimetric resonant reflectance biosensor surface, a dielectric film stack biosensor surface, or a grating-based waveguide biosensor surface;
(b) contacting the cells, cell aggregates, or tissue with the test toxin;
(c) detecting periodic or continuous peak wavelength values or effective refractive index values during a time course of the assay;
(d) analyzing the peak wavelength values or effective refractive index values for frequency, amplitude, or kinetic profile or a combination thereof over the time course of the assay to generate a signature kinetic profile of the test toxin's effects on the cells, cell aggregates, or tissue; and
(e) comparing the signature kinetic profile of the test toxin to signature kinetic profiles of known toxins, wherein the test toxin is placed into a class or subclass of toxins having a similar signature kinetic profile as the test toxin.

17. A method for determining the effect of a test compound or environmental condition on the sinus rhythm of cardiomyocytes compromising:
(a) applying the cardiomyocytes to a colorimetric resonant reflectance biosensor surface, a dielectric film stack biosensor surface, or a grating-based waveguide biosensor surface;
(b) contacting the cardiomyocytes with the compound or environmental condition;
(c) detecting periodic or continuous peak wavelength values or effective refractive index values during a time course;
(d) analyzing the peak wavelength values or effective refractive index values for sinus rhythm over the time course;
wherein a change in the sinus rhythm after the compound or environmental condition is contacted with the cardiomyocytes indicates that the compound or environmental condition has an effect on the sinus rhythm of the cardiomyocytes.

18. The method of claim 17, wherein the effect of the test compound or environmental condition on the sinus rhythm is a prolongation or shortening of the QT interval.

19. A method for determining a beat or burst pattern of cardiac or neuronal cells, cardiac or neuron cell aggregates, or cardiac or neuronal tissue comprising:
(a) applying the cells, cell aggregates, or tissue to a colorimetric resonant reflectance biosensor surface, a dielectric film stack biosensor surface, or a grating-based waveguide biosensor surface;
(b) detecting periodic or continuous peak wavelength values or effective refractive index values during a time course;
(c) analyzing the peak wavelength values or effective refractive index values for frequency, amplitude, or kinetic profile, or a combination thereof over the time course;
wherein a beat or burst pattern of the cardiac or neuronal cells, cardiac or neuron cell aggregates, or cardiac or neuronal tissue is determined.

20. The method of claim 19, wherein one or more compounds are added to the cells, cell aggregates, or tissue before or after they are applied to the biosensor surface.
